(19) United States
(12) Patent Application Publication
(10) Pub. No.: US 2013/0006052 A1
(43) Pub. Date: Jan. 3, 2013

(54) POPUP TYPE ENDOSCOPE PROVIDING THREE-DIMENSIONAL IMAGE DATA

(75) Inventor: Kwang-Seog Song, Seoul (KR)

(73) Assignees: MGB ENDOSCOPY CO.,LTD., Seoul (KR); Kwang-Seog SONG, Seoul (KR)

(21) Appl. No.: 13/635,676

(22) PCT Filed: Mar. 4, 2011
(86) PCT No.: PCT/KR2011/001504

§ 371 Date: Sep. 17, 2012

(30) Foreign Application Priority Data
Mar. 16, 2010 (KR) 10-2010-0023253

(51) Int. Cl. A61B 1/05; A61B 1/005

(52) U.S. Cl. 600/109

(57) ABSTRACT

This invention is of the pop-up endoscope providing 3-dimensional image data which provides 3-dimensional image through a pair of filming parts stored within the fore-end and projects popping-up to spread out. It allows minimizing the incision during surgery. This invention allows a pair of CCD cameras which can visualize more effective 3-dimensional image to be inserted into the patient's body and minimize the incision owing to relatively wide distance between elements.

### TECHNICAL FIELD

This invention is pop-up endoscope providing 3-dimensional image data with camera pickup element, which is able to minimize the size of the incision during surgery.

### BACKGROUND ART

Typically, endoscope consists of a single tube called direct endoscope. It can be inserted into a patient's body (i.e. inserted through mouth, or through the incision site in surgery. Insertion here refers to the latter for convenience's sake) to observe the inside for treatment and diagnosis so that the doctor can see organs with the naked eye during treatment.

However, this type of endoscope in a single tube provides 2D (2-dimensional, flat) image resulting in lack of cubic effect. This has been causing difficulty in a complete access to the patient's treatment location (the affected area).

As a solution for this problem, we as an applicant have applied for a patent of ‘the endoscope providing 3-dimensional image data’ (application no. 10-2007-0096119) on Sep. 20, 2007. The endoscope described above, as shown in FIG. 1, for the endoscope providing image data while inserted into the patient's body, features a pair of lens parallelized on the front of endoscope; a pair of CCD cameras at the back of the lens pair filming the image on the lens; illuminator on the front exposing light to the effected area.

‘The endoscope providing 3-dimentional image data’ mentioned above, though, has to be inserted into the stomach with the 2 array of Rod (2 cameras) hence, the incision size must be larger than with one array of Rod. In addition, when minimizing the incision of the stomach (approximately 12 mm), the distance between the two lens is too narrow to focus properly.

### DISCLOSURE

### Technical Problem

This invention is to solve the problems above by providing pop-up endoscope which produces 3-dimensional image data. It requires the minimum of insertion space when inserting into the body (minimizing the incision in surgery) with a pair of camera pickup elements being stored in the fore-end and projecting in pop-up manner while providing 3-dimensional image at the same time.

### Technical Solution

To solve the problems described above, the pop-up endoscope (100) providing 3-dimensional image data for this invention has tube frame (130) inserted into the patient's body to provide image data. It consists of the fore-end (110) with the front end open, the bending (120) which is connected to the fore-end (110) and bends when inserted into the patient's body, the first filming part (140) which is installed on a side within the fore-end (110), associated with the operation of the first operational rod to operate up and down, at the direction of rotation, with the filming element (141) installed on the front, and the second filming part (150) installed on the other side within the fore-end (110), associated with the operation of the second operational Rod connected to the bottom to operation up and down, at the direction of rotation, with the filming element (141) installed on the front.

Additionally, the illuminator (160) is installed on the central axis within the fore-end (110), associated with the operation of the third operational Rod (161) connected to the bottom to operate up and down, with more than one lighting installed on the front.

Also, the first operational rod (142), the second operational Rod (151), and the third operational Rod (161) are composed of elastic material.

On the other hand, in the pop-up endoscope providing 3-dimensional image data based on another implementation example for this invention, for the endoscope with a pipe-form frame (130) providing image data within the patient's body, the tube frame (130) features the fore-end (110) forming the illuminator (160) at the front end, the bending (120) which is connected to the fore-end (110) and bends when the fore-end (110) is inserted into the patient's body; the first filming part (140) with the first operational rod (142) connected on one side installed through the first slit formed on one side of the fore-end (110) to operate up and down, at direction of rotation, with the filming element (141) installed on the front; the second filming part (150) with the second operational Rod (151) on one side installed through the second slit on the other side of the fore-end (110), associated with the operation of the second operational Rod (151) to operate up and down, at direction of rotation, with the filming element (141) installed on the front, located between the fore-end (110) and the first filming part (140) when stored.

### Advantageous effects

As described above, this invention can broaden the filming element interval to minimize the incision while inserting a pair of CCD cameras that can visualize more effective 3-dimensional image.

In addition, it can adjust the angle and scope of filming only by controlling the angle of the fore-end without changing the angle of the endoscope frame inserted.

### DESCRIPTION OF DRAWINGS

FIG. 1 shows the structure of existing endoscope providing 3-dimensional image data.

FIG. 2 shows the appearance of the pop-up endoscope providing 3-dimensional image data based on recommended application of this invention.

FIG. 3 shows the cross section of the pop-up endoscope providing 3-dimensional image data with the first filming part (140) and the second filming part (150) projecting and spreading out based on recommended application of this invention.

FIG. 4 shows the cross section of the pop-up endoscope providing 3-dimensional image data with the illuminator (160) projecting and spreading out based on recommended application of this invention.

FIG. 5 shows the operational process of the pop-up endoscope providing 3-dimensional image data based on recommended application.

FIG. 6 shows the service condition of pop-up endoscope providing 3-dimensional image data based on recommended application.

FIG. 7 shows the operational process of the pop-up endoscope providing 3-dimensional image data base on another implementation example for this invention.

FIG. 8 shows the appearance during the usage of the pop-up endoscope providing 3-dimensional image data base on another implementation example for this invention.

FIG. 9 shows the status during the usage of the pop-up endoscope providing 3-dimensional image data base on another implementation example for this invention.

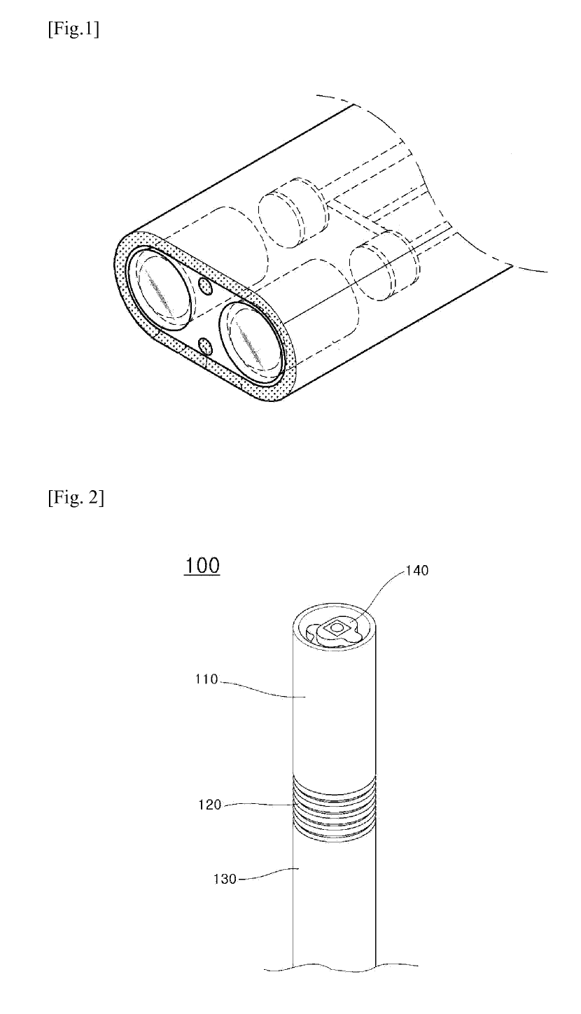

### MODES FOR CARRYING OUT THE INVENTION

The pop-up endoscope providing 3-dimensional image data based on one implementation example for this invention is described in detail below referring to the figures attached. First, note that the same elements and parts among the figures represent the same reference marks. For the description of this invention, details on the announced technologies or configuration related are omitted to clarify the points of this invention.

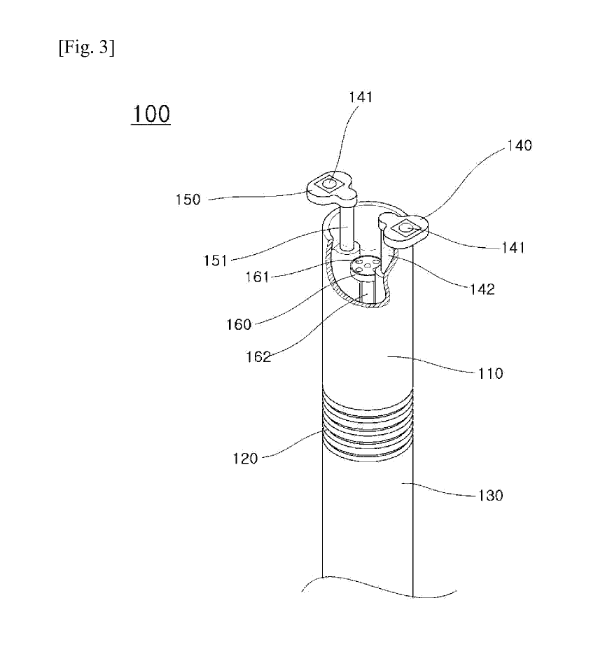

This invention contains the fore-end (110), the bending (120), the frame (130), the first filming part (140) and the second filming part (150) as shown in the FIGS. 2 and 3.

First, the fore-end (110) is open at the front end and has the space to store the first filming part (140) and the second filming part (150) inside, as shown in the FIG. 2.

Next, the bending (120) is connected to the back of the fore-end (110) allowing the fore-end (110) to bend at required angle against the frame (130) when inserted into the patient's body as shown in the FIG. 2. There are a variety of mechanical configurations available for the bending (120). Details on this configuration are omitted since it is a part of the announced technologies in the field of this invention.

The first filming part (140) is stored on one side within the fore-end (110) in stored position as shown in the FIG. 2. At the bottom of the first filming part, as shown in the FIG. 3, there connected the first operational rod (142) so that it can move up and down, at direction of rotation as the first operational rod (142) moves. In addition, on the front of the first filming part (140), as shown in the FIG. 3, there installed the filming element (141). The filming element (141) may contain lens and CCD (Charge Coupled Device). In this case, the lens is desired to have zoom and tilt functions. The details on the technologies composing the filming element (141) using the CCD (Charge Coupled Device) and lens and those on zoom/tilt functions are omitted since they are announced technologies in this field.

On the other hand, the second filming part (150) is stored on the other side within the fore-end (110) in stored position below the first filming part (140) as shown in the FIG. 2. At the bottom of the second filming part, as shown in the FIG. 3, there connected the second operational rod (151) so that it can move up and down, at direction of rotation as the second operational rod (151) moves. In addition, on the front of the second filming part (150), as shown in the FIG. 3, there installed the filming element (141).

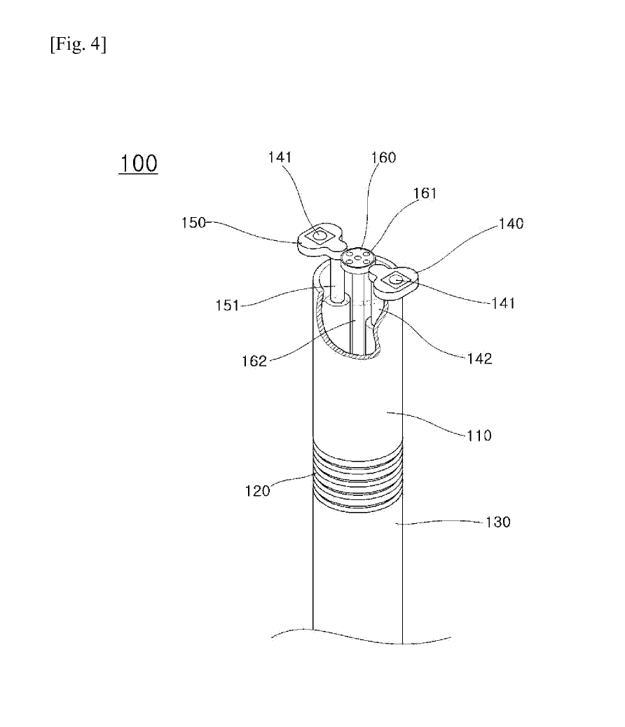

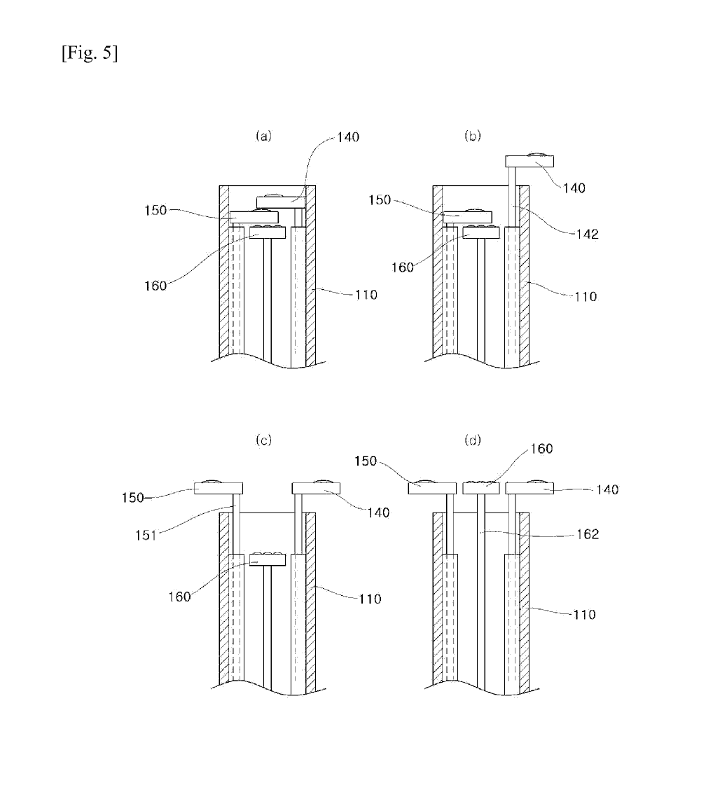

The illuminator (160) is located below the second filming part (150) on the central axis within the fore-end (11) in stored position as shown in the FIG. 5a. The third operational Rod (161) are connected at the bottom of the illuminator (160) allowing the illuminator to operate up and down, at direction of rotation as the third rod (161) move. When used within the patient's body, more than one illumination should be installed on the front of the illuminator (160) as shown in the FIG. 4 in order to throw a light on the affected area. There are a variety of configurations available such as LED (Light Emitting Diode), optical fiber connected to the light source externally installed through the frame (130). The details on the technologies composing this lighting (162) are omitted since they are the announced technologies in the field.

On the other hand, the first operational rod (142), the second operational rod (151), and the third operational rod (161) are desired to be composed of elastic materials, considering the bending movement of the bending (120). The first operational rod (142), the second operational rod (151), and the third operational rod (161) are connected to the outside through the inside of the frame to operate according to the user's control.

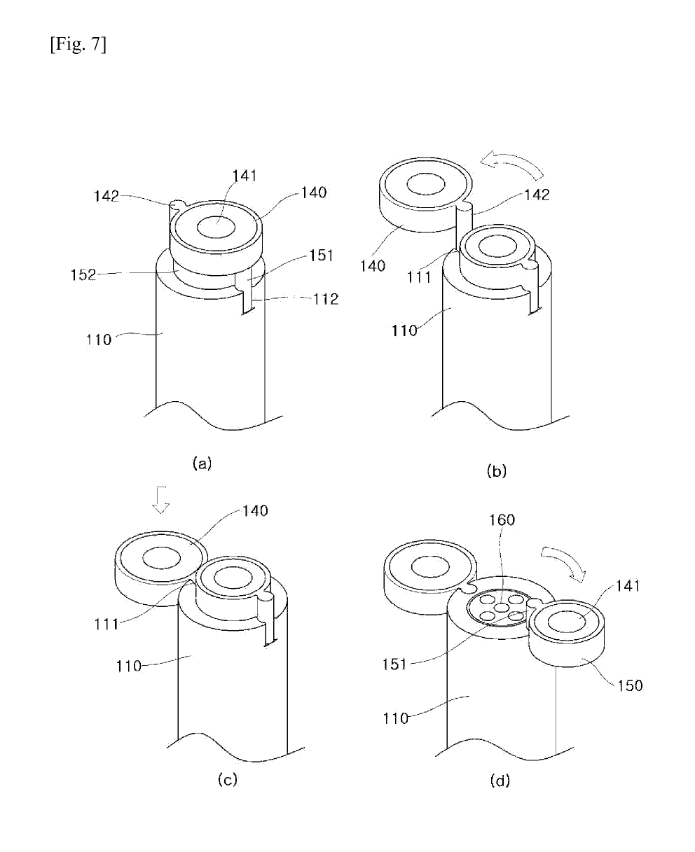

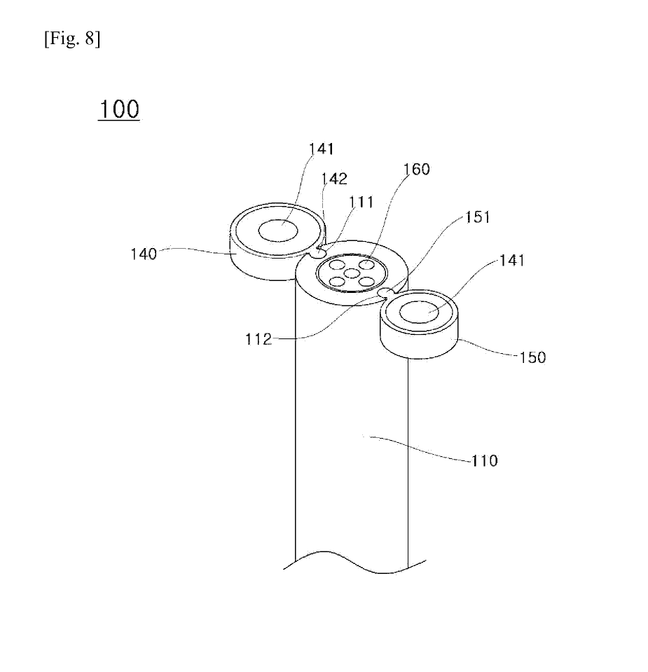

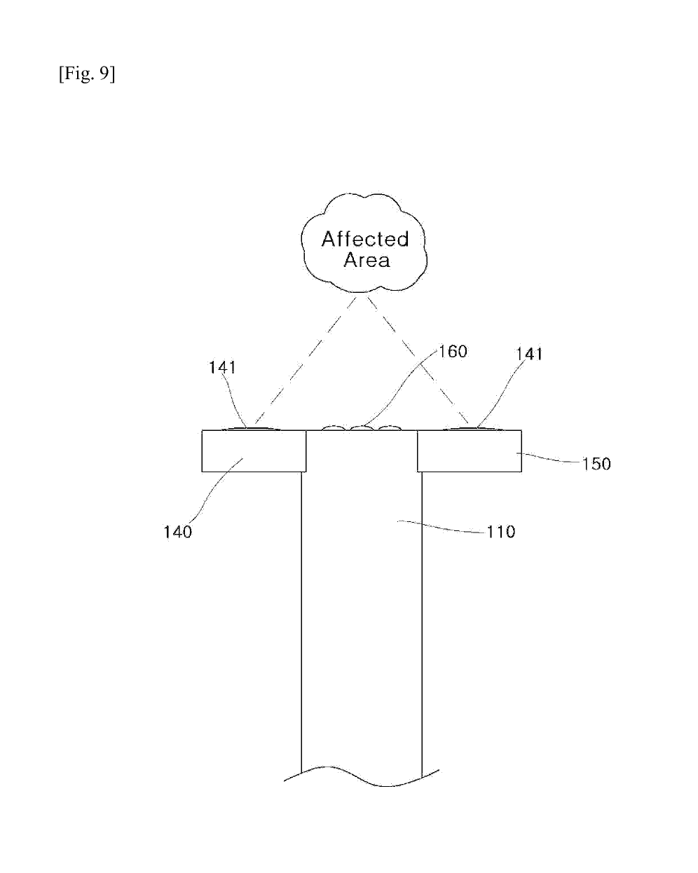

Also, for the pop-up endoscope providing 3-dimensional image data base on another implementation example, as shown in the FIGS. 7 and 8, the form and the location of the first filming part (140), the second filming part (150), and the illuminator (160) can be different. In this case, the illuminator is desired to be installed at the front end of the fore-end (110) as shown in the FIGS. 7 and 8. In addition, for the first filming part (140), it is desired that the first operational rod (142) be connected on one side installed passing through the first slit (111) on one side of the fore-end (110) as shown in the FIGS. 7 and 8, so that it can operate up and down, at direction of rotation as the first operational rod (142) moves. For the second filming part (150), it is desired that the second operational rod (151) be connected on the other side installed passing through the second slit (112) on the other side of the fore-end (110) as shown in the FIGS. 7 and 8, so that it can operate up and down, at direction of rotation as the second operational rod (151) moves. In this case, the second filming part (150) is located between the fore-end (110) and the first filming part (140) in the stored position as shown in the FIG. 7a.

For realizing the pop-up endoscope providing 3-dimensional image data based on another implementation example for this invention, the form of the fore-end (110), the first filming part (140), the second filming part (150), the first operational rod (142) and the second operational rod (151) can be cylindrical. In this case, if the diameter of the fore-end (110) is R0, the first filming part (140) R1, the second filming part (150) R2, the first operational rod (142) and the second operational rod (151) R3, the desired relationships between R0˜R3 is as follows for smooth operation.

R1<R0-R3

R2<R1-R3

The operation of the pop-up endoscope (100) providing 3-dimensional image data based on one implementation example for this invention is described below.

First, in stored position, as shown in the FIG. 5a, the first filming part (140), the second filming part (150) and the illuminator (160) are stored in the order within the fore-end (110) from the outside. And then, the fore-end (110), the bending (120) and the frame (13) are inserted into the patient's body through the incision.

After that, as shown in the FIG. 5b, the first filming part (140) projects outward from the fore-end (110) by the operation of the first operational rod (142) and then spreads outward from the extension of the border of the fore-end (110) by the rotation of the first operational rod(142).

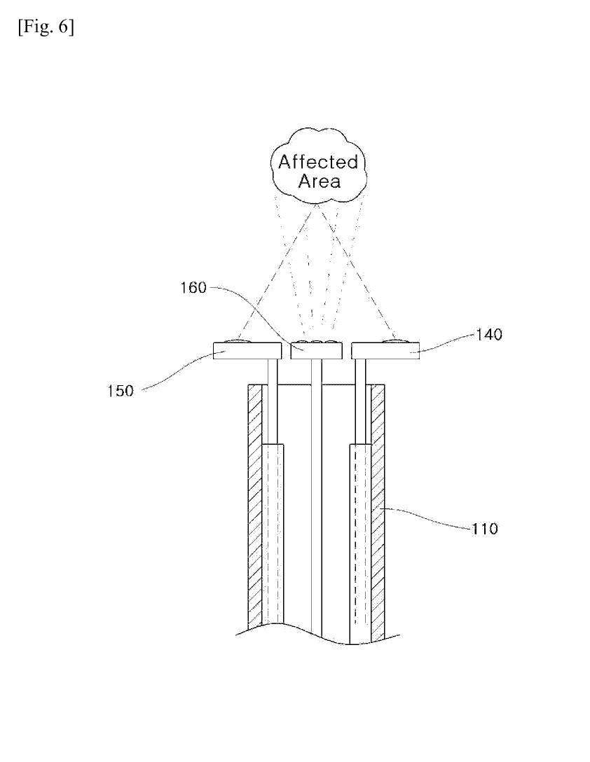

Next, as shown in the FIG. 5c, the second filming part (150) projects outward from the fore-end (110) by the operation of the second operational rod (151) and then spreads outward from the extension of the border of the fore-end (110) by the rotation of the second operational rod (151). The pop-up projection/spreading operation of the first filming part (140) and the second filming part (150) allows them to project and spread out exceeding the diameter of the frame (130) even in operating status as shown in the FIG. 6. It minimizes the width of the incision and achieves effective 3-dimensional image.

On the other hand, in the case where the illuminator (160) is contained, as shown in the FIG. 5d, the illuminator (160) projects from the fore-end (110) by the operation of the third operational rod (161), locating itself between the first filming part (140) and the second filming part (150) to illuminate the affected area as shown in the FIG. 6.

After using the pop-up endoscope providing 3-dimensional image data based on one implementation example for this invention, the illuminator (160), the second filming part (150), and the first filming part (110) are stored in the order within the fore-end (110) in reverse order of the projection/spreading process. Therefore, there is no projected structure in the scope exceeding the external diameter of the frame (130) including the fore-end (110) making pulling out the fore-end (110) and the frame (130) out of the minimized incision easier.

On the other hand, for the pop-up endoscope providing 3-dimensional image data based on another implementation example for this invention, the operation proceeds in the same order as shown in the FIG. 7.

First, the first filming part (14), the second filming part (15) and the fore-end (110) are inserted into the patient's body folded on the top of one another as shown in the FIG. 7a.

Then, the first filming part (140) rotates outward from border of the fore-end (110) to spread out as the first operational rod (142) operates as shown in the FIG. 7b. Next, it descends to the same height as the top of the fore-end (110) as the first operational rod (142) descends as shown in the FIG. 7c. In this case, the link between the first filming part (140) and the first operational rod (142) locates itself as if it is inserted into the first slit (111).

Next, the second filming part (150) rotates outward from border of the fore-end (110) to spread out as the second operational rod (151) operates as shown in the FIG. 7d. Next, it descends to the same height as the top of the fore-end (110) as the second operational rod (151) descends as shown in the FIG. 8. In this case, the link between the second filming part (150) and the second operational rod (151) locates itself as if it is inserted into the second slit (112).

After using the pop-up endoscope providing 3-dimensional image data based on another implementation example for this invention, the second filming part (150) and the first filming part (110) are stored again in the order within the fore-end (110) in reverse order of the projection/spreading process. Therefore, there is no projected structure in the scope exceeding the external diameter of the frame (130) including the fore-end (110) making pulling out the fore-end (110) and the frame (130) out of the minimized incision easier.

The ideal implementation examples are introduced above. Although specific terms are used here, it is for the purpose to only describe this invention, not to limit the extent of this invention written in the scope of the request for the patent. Thus, those who have the common knowledge in the technological field would understand that this invention is able to transform to various forms and equivalent implementation examples. The technological protection measures should be planned based on the technological ideology in the scope of the patent request attached.

1. An endoscope having a pipe frame inserted into a patient's body to provide image data, wherein the pipe frame comprises
a fore-end whose front end is open;
a bending which is connected to the fore-end and bends when the fore-end is inserted into the patient's body;
a first filming part which is installed on one side within the fore-end, operates to move up and down and to rotate as a first operational rod coupled at a lower end operates, and of which a filming element is installed on the front; and
a second filming part which is installed on the other side within the fore-end, operates to move up and down and to rotate as a second operational rod coupled at the lower end operates, and of which the filming element is installed on the front.

2. The endoscope to provide image data according to claim 1, wherein the pipe frame further comprises an illuminator which is installed on a central axis within the fore-end, operates up and down as a third operational rod coupled at the lower end operates, and of which more than one lighting is installed on the front.

3. The endoscope to provide image data according to claim 2, wherein the first operational rod, the second operational rod, and the third operational rod are made of an elastic material.

4. An endoscope having a pipe frame inserted into a patient's body to provide image data, wherein the pipe frame comprises
a fore-end having an illuminator at its front end;
a bending which is connected to the fore-end and bends when the fore-end is inserted into the patient's body;
a first filming part of which a first operational rod is connected on one side to be installed passing through a first slit formed on one side of the fore-end, and which can operate up and down and rotate as the first operational rod operates, and which has an filming element installed on the front; and
a second filming part of which a second operational rod is connected on one side to be installed passing through a second slit formed on one side of the fore-end, and which can operate up and down and rotate as the second operational rod operates, and which has a filming element installed on the front.
